(19) United States
(12) Patent Application Publication
(10) Pub. No.: US 2016/0115225 A1
(43) Pub. Date: Apr. 28, 2016

(54) Methods of Reducing Methylglyoxal (MGO) Modification of Recombinant Proteins in Cell Culture

(71) Applicant: AbbVie Inc., North Chicago, IL (US)

(72) Inventors: Christopher M. Chumsae, North Andover, MA (US); Kartik Subramanian, Northborough, MA (US); Yun ("Frank") Zhang, Shrewsbury, MA (US)

(73) Assignee: AbbVie Inc.

(21) Appl. No.: 14/922,088

(22) Filed: Oct. 23, 2015

Related U.S. Application Data
(60) Provisional application No. 62/068,328, filed on Oct. 24, 2014.

(51) Int. Cl. C07K 16/24

(52) U.S. Cl. CPC C07K 16/241; C07K 2317/14

(57) ABSTRACT

Methods are provided for controlling the levels of acidic species when a protein of interest is expressed by host cells. One particular example of an acidic species is methylglyoxal (MGO). Reduction of acidic species such as MGO may be achieved by regulating the glucose consumption of the cell culture or by modulating the cysteine/glutathione metabolic pathways.

### CROSS-REFERENCE TO RELATED APPLICATION

The present application claims the benefit of U.S. Provisional Application No. 62/068,328, filed Oct. 24, 2014, the disclosure of which is incorporated by reference in its entirety.

### BACKGROUND OF THE INVENTION

1. Field of the Invention

The present invention relates to protein production. More particularly, it relates to compositions and processes for controlling the amount of acidic species generated during recombinant expression of a protein. Even more particularly, it relates to compositions and processes for reducing the amount of methylgloxyl (MGO) in recombinant proteins produced by cell culture, such as the antibody adalimumab.

2. Description of Related Art

The production of proteins for biopharmaceutical applications typically involves the use of cell cultures that are known to produce proteins exhibiting varying levels of post-translational modifications that contribute to heterogeneity. Such heterogeneity includes, but is not limited to, the presence of acidic species heterogeneity. For example, in the preparation of monoclonal antibodies (mAbs), such acidic species heterogeneities can usually be detected by various methods, such as WCX-10 HPLC (a weak cation exchange chromatography) or IEF (isoelectric focusing). In one example, the acidic species identified using such techniques may encompass a range of product-related impurities, such as antibody product fragments (e.g., Fc and Fab fragments), and/or post-translation modifications of the antibody product such as deamidated or glycoslyated antibodies.

Under certain cell culture conditions, specific modification of recombinant proteins by the metabolite methylglyoxal (MGO) have been reported (Chumsae, C. et al., Anal. Chem. 85, 11401-11409 (2013)). Such modifications cause the antibody to elute earlier and within the acidic species part of the WCX-10 chromatogram. The degree of shift in retention time in the chromatogram is dependent on the extent of modification that is likely to happen on multiple sites, primarily arginine residues, on the antibody. As a consequence of this modification, about a three-fold reduction in TNF-alpha binding efficiency of a MGO enriched fraction of adalimumab in comparison to an unmodified main product fraction of adalimumab has been observed. See e.g., U.S. patent application Ser. No. 14/078,181, which is incorporated by reference into this disclosure.

### BRIEF SUMMARY OF THF INVENTION

In one embodiment, the present disclosure provides methods to control (modulate or reduce) acidic species heterogeneity in a population of proteins. In particular, the presence of such acidic species corresponds to heterogeneity of the distribution of charged impurities due to methylglyoxal (MGO) modification of the proteins. This heterogeneity is of particular interest when it arises in the context of recombinant protein production.

In certain embodiments, control may be exerted over the amount of acidic species of protein produced by cell culture. In particular, control may be exerted over the extent of MGO modification of the protein produced. This corresponds to a combination of several such MGO modifications at multiple residues or sites on the population of proteins produced by cell culture. In certain embodiments, the control may be exerted over the amount of acidic species or MGO modification formed while the protein is present in a cell culture broth, while the culture is actively maintained, or while the cells are removed. In certain embodiments, the protein may be an antibody, such as adalimumab.

In certain embodiments, control over the amount of acidic species or MGO modification produced by cell culture may be exerted by modulating the rate of glucose consumption during cell culture. In certain embodiments, the rate of glucose consumption for control of acidic species generation or MGO modification may be modulated during production of a protein of interest, for example, an antibody, such as adalimumab. In certain embodiments, reduction of the amount of acidic species formation or MGO production or MGO modification of protein produced by cell culture may be exerted by reducing the glucose consumption rate of the cells. In certain embodiments, the control may be influenced right from the beginning of culture and in other cases throughout the course of the culture.

In certain embodiments, the reduction of glucose consumption for reduction in MGO production or MGO modification of the protein or acidic species of the protein may be exerted by supplementing the media of cells expressing the protein of interest with glycolysis inhibitors, for example, by supplementing the media with 2-Deoxy-D-Glucose (2DDG). In certain embodiments, the concentration of 2DDG to be supplemented to the cell culture media may range from about 0.05 g/l to 0.5 g/l, or from about 0.15 g/l to 0.25 g/l, at a concentration of about 0.15 g/l or about 0.25 g/l.

In certain embodiments, the reduction of glucose consumption for reduction in MGO production or MGO modification of protein or acidic species of the protein may be exerted by controlling the concentration of glucose during cell culture. For example, in certain cases this may include modulating glucose feed amounts to target the glucose concentration in a range of 0-2 g/l. In certain embodiments, this may be implemented in certain phases of culture, for example, the post initial growth phase.

In certain embodiments, control over the extent of MGO production or MGO modification of the protein may be exerted by employing a temperature shift, for example, by shifting the temperature in the bioreactor from 35° C. to 33° C. on Day 5 of culture. In another embodiment, the shifting the temperature occurs after Day 3, or Day 4 of culture. In certain embodiments, reduction in MGO modification or acidic species of the protein obtained through the temperature shift also corresponds to the reduction of glucose consumption post temperature shift.

In certain embodiments, control over the formation of acidic species or the extent of MGO production or MGO modification of protein may be exerted by the combination of methods employed to reduce glucose consumption in combination with supplementation of the basic amino acid arginine to cell culture, and lowering the pH of the bioreactor to a pH of 6-6.9.

In certain other embodiments, control over the formation of acidic species or the extent of MGO production or MGO modification of protein may be exerted by the combination of methods employed to reduce glucose consumption in combination with supplementation of the amino acid lysine to cell culture, and lowering the pH of the bioreactor to a pH of 6-6.9.

In certain embodiments, control over the formation of acidic species or the extent of MGO production or MGO modification of protein may be exerted by the combination of methods employed to reduce glucose consumption in combination with supplementation of the amino acid histidine to cell culture, and lowering the pH of the bioreactor to a pH of 6-6.9.

In certain embodiments, a reduction in MGO modification or acidic species of the protein obtained by lowering bioreactor culture pH also corresponds to a reduction of glucose consumption.

In certain embodiments, control over the extent of MGO production or MGO modification of protein may be exerted by supplementation of the amino acid serine. For example, in certain cases this supplementation may include supplementation of serine in the range of 0.1-5 g/l or 0.1-2 g/l. In certain embodiments, the supplementation of serine may be combined with one or several of the following vitamins cocktail: vitamin B6, vitamin B2, vitamin B12, Nicotinamide, folate, and choline. In another embodiment, the cell culture may be supplemented with at least one vitamin selected from the group consisting of vitamin B6, vitamin B2, vitamin B12, Nicotinamide, folate, choline, and combinations thereof. In certain cases, this reduction in MGO modification obtained through these supplementations also corresponds to modulation of homocysteine, cysteine, or glutathione metabolite levels.

### BRIEF DESCRIPTION OF THE DRAWINGS

FIG. 1 depicts the effect of 2DDG supplementation and temperature shift (35° C. to 33° C. on day 5) in bioreactor batch culture with cell line 1 on (a) cell growth and (b) viability, as described in Example 2.

FIG. 2 depicts the effect of 2DDG supplementation and temperature shift (35° C. to 33° C. on day 5) in bioreactor batch culture with cell line 1 on titer (normalized to the control), as described in Example 2.

FIG. 3 depicts the effect of 2DDG supplementation and temperature shift (35° C. to 33° C. on day 5) in bioreactor batch culture with cell line 1 on percentage of acidic species, as described in Example 2.

FIG. 4 depicts the effect of 2DDG supplementation and temperature shift (35° C. to 33° C. on day 5) in bioreactor batch culture with cell line 1 on (a) percentage of MGO modified peptides (out of those that can be modified) and (b) specific glucose consumption rate, as described in Example 2.

FIG. 5 depicts the effect of combination of 2DDG supplementation and temperature shift (35° C. to 33° C. on day 5) with arginine supplementation and lower bioreactor final pH in bioreactor batch culture with cell line 1 on (a) percentage of acidic species and (b) percentage of MGO modified peptides (out of those that can be modified), as described in Example 3.

FIG. 6 depicts the effect of combination of 2DDG supplementation and temperature shift (35° C. to 33° C. on day 5) along with arginine supplementation and lower bioreactor final pH in bioreactor batch culture with cell line 1 on (a) extracellular MGO production rate (day 7 to day 8) of culture and (b) cumulative glucose consumption up to day 7 of culture, as described in Example 3.

FIG. 7 depicts the correlation between cumulative glucose consumption (up to day 7) and extracellular MGO concentration (day 7) for all the tested conditions of the DOE experiment, as described in Example 3.

FIG. 8 depicts the effect of 2DDG supplementation in bioreactor batch culture with cell line 2 on (a) glucose concentration profile and (b) extracellular MGO concentration profile, as described in Example 4.

FIG. 9 depicts the effect of 2DDG supplementation in bioreactor batch culture with cell line 2 on the correlation between specific glucose consumption and specific extracellular MGO production, as described in Example 4.

FIG. 10 depicts the effect of 2DDG supplementation in bioreactor batch culture with cell line 2 on percentage of acidic species, as described in Example 4.

FIG. 11 depicts the effect of glucose feed control strategy in bioreactor batch culture with cell line 2 on (a) glucose concentration profile and (b) extracellular MGO concentration profile, as described in Example 4,

FIG. 12 depicts the effect of glucose feed control strategy in bioreactor batch culture with cell line 2 correlation between specific glucose consumption and specific extracellular MGO production, as described in Example 4.

FIG. 13 depicts the effect of glucose feed control strategy in bioreactor batch culture with cell line 2 on percentage of acidic species, as described in Example 4.

FIG. 14 depicts the effect of amino acid serine and vitamin supplementation in shake-flask batch culture with cell line 2 on (a) cell growth and (b) viability, as described in Example 5.

FIG. 15 depicts the effect of amino acid serine and vitamin supplementation in shake-flask batch culture with cell line 2 on (a) free extracellular MGO concentration and (b) percentage of MGO modified peptides (out of those that can be modified), as described in Example 5.

FIG. 16 depicts the effect of amino acid serine and vitamin supplementation in shake-flask batch culture with cell line 2 on relative abundance of cysteine, homocysteine and glutathione, as described in Example 5.

### DETAILED DESCRIPTION OF THE INVENTION

This disclosure provides methods and compositions for reducing the overall levels of acidic species variants on recombinant proteins. In one aspect of the disclosure, the acidic species reduction is related particularly to the reduction of methylglyoxal (MGO) modification, which is a particular variant within the entire acidic species. In another aspect of this disclosure, controlling the amount of MGO modification generated during expression of a protein of interest by host cells is achieved by regulating the glucose consumption of the cell culture. This may be achieved by modifying the culture media of the cells or by manipulating the process parameters during the cell culture operation. In another aspect of the disclosure, controlling the amount of MGO modification is also achieved through modulation of the cysteine/glutathione synthesis pathway. In certain embodiments, this is achieved through supplementation of the cell culture media with the amino acid serine along with a cocktail of vitamins.

In one embodiment, the methods and compositions disclosed herein are suitable for reduction of acidic species during the expression of the anti-INF alpha antibody adalimumab as disclosed in U.S. Pat. No. 6,090,382 and in U.S. patent application Ser. No. 14/078,181, which are hereby incorporated by reference into the present disclosure.

MGO may be generated as a three carbon byproduct of glycolysis, lipid catabolism and amino acids threonine and glycine breakdown (Chaplen, F. W. R., Cytotechnology 26: 173-183 (1998)). Several studies have summarized the biochemical pathways involved in the breakdown and elimination of MGO. Id. One such pathway is the glyoxalase pathway where MGO is metabolically broken down into D-Lactate in a glutathione-mediated way. Another pathway is the reduction of MGO to propanediol through the enzyme aldose reductase. Evidently, under non-optimal operation of these metabolic pathways, MGO may be allowed to accumulate in cell cultures, especially under conditions of decreasing cell viability, increasing the risk of protein modification. In one embodiment, one of the objectives is to devise cell culture methods that control either the synthesis of MGO and/or enhance the mechanism for its elimination.

It has been shown that supplementation of the basic amino acid arginine to cell culture media and bioreactor operation at lower culture pH are strategies to help reduce the extent of MGO modification (WO 2013158275A1). Since MGO is a byproduct of glycolysis, researchers studying elevated blood glucose levels in diabetes have reported protein modifications by MGO. The rate of MGO formation in normal cell culture systems has been estimated to be at 0.4-0.5% of the glycolytic flux. Due to the high glucose concentration in industrial cell culture media (up to 50 mM) and the significantly high glycolytic flux, significant levels of MGO have been reported (Chaplen, F. W. R., Cytotechnology 26:173-183 (1998)). In other studies, no discernible differences in glucose levels were observed between mammalian culture condition samples that had substantially different levels of MGO modification (Chumsae, C. et al., Anal. Chem. 85: 11401-11409 (2013)).

Recent reports have also suggested that increasing amounts of MGO are synthesized as a function of time in mammalian cell culture starting in the phase preceding the phase of cell death or decreasing viability (Paoli, T. et al., Biotechnology and Bioengineering 107: 182-189 (2010)). It was previously unknown whether overall MGO production and consequently MGO modification of proteins can be reduced by modulation of cell culture conditions. It was also not known whether a suitable feedback control strategy may be used to regulate MGO production.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well-known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The heavy chain variable region (domain) is also designated as VDH in this disclosure. The CH is comprised of three domains, CH1, CH2 and CH3. Each light chain is comprised of a light chain variable region (VL) and a light chain constant region (CL). The CL is comprised of a single CL domain. The light chain variable region (domain) is also designated as VDL in this disclosure. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass. In certain embodiments, the antibody is adalimumab.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. Coli; mammalian cell lines CHO, HEK293; COS, NS0, SP2 and PER.C6; the insect cell line Sf9; and the fungal cell Saccharomyces cerevisiae.

Various modifications of the proteins may occur during recombinant expression of a protein of interest. The difference in post-translational modification may be effected by addition of one or more chemical groups to the amino acids of the modified molecule, or removal of one or more such groups from the molecule. Examples of modification may include but are not limited to, phosphorylation, glycosylation, or MGO modification.

“Glycolysis” refers to the metabolic pathway in which glucose is converted into pyruvate.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### Examples

### Example 1

### Materials and Methods

### 1. Cell Source and Adaptation Cultures

Two adalimumab producing cell lines were employed in the studies covered here. Upon thaw, cells were cultured in their respective growth media in a combination of vented non-baffled shake flasks (Corning) on a shaker platform at 110 RPM (cell line 1) and 140 RPM (cell line 2) and 10 L or 20 L wave bags (GE). Cultures were propagated in a 35° C., 5% CO₂ incubator for cell line 1 and 36° C., 5% CO₂ incubator for cell line 2 to obtain the required number of cells to be able to initiate production stage cultures.

### 2. Cell Culture Media

Growth and production media were prepared from a chemically defined media formulation (IVGN CD media). For preparation of the IVGN CD media formulation, the media (IVGN GIA-1) was supplemented with L-glutamine, sodium bicarbonate, sodium chloride, and methotrexate solution. Production media consisted of all the components in the growth medium, excluding methotrexate. For cell line 1, both growth and production medium were also supplemented with insulin.

2-Deoxy-D-Glucose (2DDG) (Sigma), amino acid Arginine (Sigma) or Serine (Sigma) and vitamins: Vitamin B6 (SAFC), Choline (SAFC), Folate (SAFC), Vitamin B2 (SAFC), Vitamin B12 (Calbiochem) and Nicotinamide (SAFC) were added as additional supplements to the cell culture media for certain experimental conditions. The 2DDG used for the experiments were either reconstituted in Milli-Q water to make a 20 g/l stock solution, that was later supplemented to the culture media or added directly as a powder during medium formulation. Arginine and Serine were supplemented directly to the media at the prescribed concentration per the experimental design. All vitamins except folate were added directly as a powder during medium formulation. A stock solution of folate was initially prepared in 1N NaOH and then subsequently added to medium formulation to achieve the specific concentration listed per the design. All media was filtered through a 0.22 μm filter and stored at 4° C. until usage.

### 3. Production Cultures

Production cultures were initiated either in 500 ml shake flasks (Corning) or in 3 L Bioreactors (Applikon).

For shake flask experiments (Example 4), duplicate 500 mL Corning vented non-baffled shake flasks (200 mL working volume) were used for each condition. Similar to the seed train stage, the production stage shake flasks were kept in incubators maintained at 36° C. and 5% CO₂ on shaker platforms that were set at 140 rpm.

For the bioreactor (3 L Applikon) experiments (Examples 1, 2 and 3), 3 L, bioreactors (1.5 L working volume) were initiated at 35° C. (cell line 1) and 36° C. (cell line 2), 30% DO, 200 rpm, pH of 7.1. In some of the conditions, pH was ramped down to 6.8 in three days and kept at pH 6.8 thereafter. The inclusion of pH ramp for a particular condition is indicated by the final pH in the experimental design. If the final pH is 7.1, then it indicates no pH ramp was performed, whereas if the final pH is 6.8, a pH ramp was performed on day 3 from pH 7.1 to pH 6.8. In some of the conditions, a temperature shift was performed. When temperature shift was performed, the temperature was lowered to 33° C. Again, the conditions where such a temperature shift was performed can be identified from the final temperature in the experimental design. Cultures were run in extended batch mode with the addition of a desired amount of 40% glucose stock solution was fed based on glucose measurements.

In all experiments, the cells were transferred from the seed train to the production stage at a split ratio of 1:5.

Retention samples for titer analysis, of 2×1.5 mL, were collected daily for the bioreactor experiments over the last few days of culture, and frozen at −80° C. The samples taken were later submitted for titer analysis.

About 20 ml of cell free supernatant were collected in some cases for measurement of extracellular MGO in the supernatant. For this, a 3,000 RPM 30 min centrifugation was performed to remove the cells.

The harvest procedure of the reactors involved centrifugation of the culture sample at 3,000 RPM for 30 min and storage of supernatant in PETG bottles at −80° C. before submission for protein A purification, WCX-10 analysis and Lys-C peptide mapping.

### 4. WCX-10 Assay

This method is employed towards the quantification of the acidic species and other charge variants present in cell culture harvest samples. Cation exchange chromatography was performed on a Dionex ProPac WCX-10, Analytical column (Dionex, Calif.). The mobile phases used were 10 mM Sodium Phosphate dibasic pH 7.5 (Mobile phase A) and 10 mM Sodium Phosphate dibasic, 500 mM Sodium Chloride pH 5.5 (Mobile phase B). A binary gradient (94% A, 6% B: 0-20 mM; 84% A, 16% B: 20-22 min; 0% A, 100% B: 22-28 min; 94% A, 6% B: 28-34 min) was used with detection at 280 nm.

Quantitation is based on the relative area percent of detected peaks. The peaks that elute at relative residence time earlier than the main peak corresponding to the drug product are together represented as the acidic peaks.

### Lysine-C Peptide Mapping for Quantification of Methylglyoxal (MGO) Modification of the Protein

Typical trypsin digestion for peptide mapping cleaves a denatured, reduced and alkylated protein at the carboxyl side of the two basic amino acids, lysine and arginine. Methylglyoxal (MGO) is a small molecule metabolite derived as a glycolysis byproduct which can modify arginine residues. A modification of an arginine prevents trypsin from cutting this site and results in a mis-cleavage. The challenge of quantifying the amount of MGO modified peptide is that, it is not compared to an equivalent non-modified peptide but rather to two parental cleaved peptides. The two parental cleaved peptides likely have different ionization potential as compared to that of the modified peptide. In order to determine a truly accurate direct measurement of an MGO-modified peptide, it is compared to its non-modified counterpart and expressed as a percentage. Using endoproteinase lysine-C as an alternative enzyme, cleavages only occur at lysine residues. The result is a direct comparison of the same peptide with and without an MGO modification, which provides a high degree of accuracy in quantifying even trace levels of the modified species.

Procedure: Samples were dilated to a nominal concentration of 4 mg/mL. 8 M guanidine-HCl was added to the sample in a 3:1 ratio resulting in a 1 mg/mL concentration in 6M guanidine-HCl. The samples were reduced with 10 mM final cone. DTT for 30 minutes at 37° C. followed by an alkylation with 25 mM final concentration iodoacetic acid for 30 minutes at 37° C. in the dark. The samples were then buffer exchanged into 10 mM Tris pH 8.0 using NAP-5 columns. The samples were then digested for 4 hours at 37° C. using endoproteinase Lys-C at an enzyme to protein ratio of 1:20. The digest was quenched by adding 5 μL of formic acid to each sample. Samples were analyzed by LC/MS peptide mapping. Briefly, 50 μL of sample was loaded onto a Waters BEH C18 1.7μ 1.0×150 mm UPLC column with 98% 0.08% formic acid, 0.02% TFA in water and 2% 0.08% formic acid, 0.02% TFA in acetonitrile. The composition was changed to 65% 0.08% formic acid, 0.02% TFA in water and 35% 0.08% formic acid, 0.02% TFA in acetonitrile in 135 minutes using a Waters Acquity UPLC system. Eluting peaks were monitored using a Thermo Scientific LTQ-Orbitrap Mass Spectrometer. Specific mass traces were extracted for both modified and non-modified peptides in order to accurately quantify the total amount of MGO modification at each site. Mass spectra were also analyzed for the specific region of the chromatogram to confirm the peptide identity.

### 6. MGO Measurement in the Cell Culture Supernatant

Remove protein: After quick thawing covered with aluminum, cell culture samples (10 mL) were filtrated by a 3 kDa or 10 kDa membrane filter (Millipore, UFC900369) until the solution left inside the filter was around 1 mL, the flow through that was used for MGO analysis.

Derivative MGO: 750 uL of flow through, 75 uL of 1 M NaH₂PO₄ buffer (pH 8), 50 mM 1,2 diaminobenzene (Aldrich P/N: P23938), and 600 uL of MilliQ water were mixed well and incubated at 60° C. for 3 hours. After cooling down and centrifuging at 10000 rpm for 3 minutes, the sample solution was ready to be injected.

External Standards to Quantitate MGO: external standards were used to quantitate MGO in cell culture. Based on the following table, the standard solutions were prepared. The standard solutions were incubated at 60° C. for 3 hours. After cooling down, the standard solutions were analyzed to get an external standard curve. The concentration of MGO in culture was calculated based on the external standard curve. (MGO: CMP Biomedicals, P/N: 155558; GO: Fisher Scientifics, P/N: S25698). 10 mM and 1 mM MGO were diluted from the MGO and 1000 uM and 10 uM GO were diluted from the GO. The diluent was water.

| | 1M | | | | | | | | |
|---|---|---|---|---|---|---|---|---|---|
| | NaH2PO4, pH | 50 mM 1, | 10 mM | 1000 uM | 3X GIA 1 | HPLC | Total | mM | uM |
| Sample ID | 8.0, uL | 2-DAB, uL | MGO, uL | GO, uL | Media, uL | Water, uL | Volume, uL | of MGO | of GO |
| B1 | 75 | 75 | 150 | 120 | 750 | 330 | 1500 | 1 | 80 |
| B2 | 75 | 75 | 120 | 90 | 750 | 390 | 1500 | 0.8 | 60 |
| B3 | 75 | 75 | 90 | 60 | 750 | 450 | 1500 | 0.6 | 40 |
| B4 | 75 | 75 | 60 | 30 | 750 | 510 | 1500 | 0.4 | 20 |
| B5 | 75 | 75 | 30 | 15 | 750 | 555 | 1500 | 0.2 | 10 |
| B6 | 75 | 75 | 15 | 9 | 750 | 576 | 1500 | 0.1 | 6 |
| | 1M | | | | | | | | |
| | NaH2PO4, pH | 50 mM 1, | 1 mM | 10 uM | 3X GIA 1 | HPLC | Total | mM | uM |
| Sample ID | 8.0, uL | 2-DAB, uL | MGO, uL | GO, uL | Media, uL | Water, uL | Volume, uL | of MGO | of GO |
| B7 | 75 | 75 | 75 | 300 | 750 | 225 | 1500 | 0.05 | 2 |
| B8 | 75 | 75 | 15 | 150 | 750 | 435 | 1500 | 0.01 | 1 |
| B9 | 75 | 75 | 7.5 | 75 | 750 | 517.5 | 1500 | 0.005 | 0.5 |
| B10 | 75 | 75 | 1.5 | 15 | 750 | 583.5 | 1500 | 0.001 | 0.1 |

HPLC Method: A reverse phase HPLC method was used for MGO analysis. The parameters are listed in the method information table and gradient table below.

### Method Information Table

| | |
|---|---|
| Mobile phase A | 0.5% Acetic acid in water |
| Mobile phase B | 100% Acetonitrile |
| Column | Zorbax 300SB-C18, 4.6 × 150 mm, 3.5 um, |
| | Agilent, P/N: 863973-902 |
| Column temperature | 50° C. |
| Flow rate | 0.6 mL/min |
| Injection volume | 20 uL |
| Detector | 313 nm |
| Auto-sampler temperature | 10° C. |

The Gradient Table

| Time, min | % Mobile Phase B |
|---|---|
| 0 | 5 |
| 5 | 25 |
| 20 | 40 |
| 22 | 100 |
| 25 | 100 |
| 26 | 0 |
| 28 | 0 |
| 29 | 5 |
| 38 | 5 |

MGO Concentration Calculation:

The measured concentration based on external quantitation multiplied by two is the final MGO concentration.

### 7. LC/MS Analysis of Cell Free Supernatant: Assessment of Free Thiol Components Disulfide Linked to Free Light Chain

The analysis of free thiol components in cell culture, i.e. cysteine, homocysteine and glutathione, was accomplished using an LC/MS analysis of clarified harvest. Briefly, the established adalimumab constructs express a surplus of light chain which is not incorporated into the assembled recombinant antibodies. This free light chain has a free thiol at Cys214, the C-terminal cysteine which would normally disulfide bond to its cognate heavy chain. The reactive nature of this thiol results in disulfide bonds with free cysteine, homocysteine or glutathione in the culture medium. An assessment of this light chain complex gave a real time relative abundance of these three species. In these studies, the levels of these components are important to the understanding of the underlying biochemistry.

Sample Prep: The clarified harvest was centrifuged in order to remove particulates and to make the sample more amenable to LC/MS analysis. The sample was then diluted to approximately 0.5 mg/mL.

LC/MS Analysis: 2-5 μL of the clarified harvest was injected onto a Vydac C4 reversed phase column (1×150 mM, 5μ particle size, 300 A) in 95% 0.08 FA/0.02 TFA in water and 5% 0.08 FA/0.02 TFA in acetonitrile at a flow rate of 50 μL/min. Gradients are as shown below.

| Time (min) | Buffer A (%) | Buffer B (%) |
|---|---|---|
| 0 | 95 | 5 |
| 5 | 65 | 35 |
| 24 | 55 | 45 |
| 24.1 | 5 | 95 |
| 27 | 5 | 95 |
| 27.1 | 95 | 5 |
| 35 | 95 | 5 |

The data was acquired on an Agilent 6510 Q-TOF mass spectrometer. The system was run in positive electrospray with a scan range of 600-3200 m/z.

### Example 2

### Effect of 2DDG Supplementation and Temperature Shift in a Bioreactor Culture with Cell Line 1

Cell line 1 was cultured in IVGN production medium supplemented with or without 2DDG (0.25 g/l). Prior to this experiment, a study was performed in a shake flask culture to evaluate the dose effect of 2DDG on this cell line and optimize a dose range of reactor evaluation (data not shown). In this experiment, the cultures were performed in 3 L bioreactors in batch format (pH 7.1, DO—30%, 200 rpm) with only glucose feed as described in Example 1. Temperature shift was implemented in some tests (35° C. to 33° C. on day 5 of culture). Both 2DDG supplementation and temperature shift were employed as methods to reduce glucose consumption of the cells in culture. The hypothesis was that the reduction in glucose consumption will translate into reduced synthesis of MGO in cell culture. As a result, a reduction in MGO modification and acidic species of the protein would be achieved.

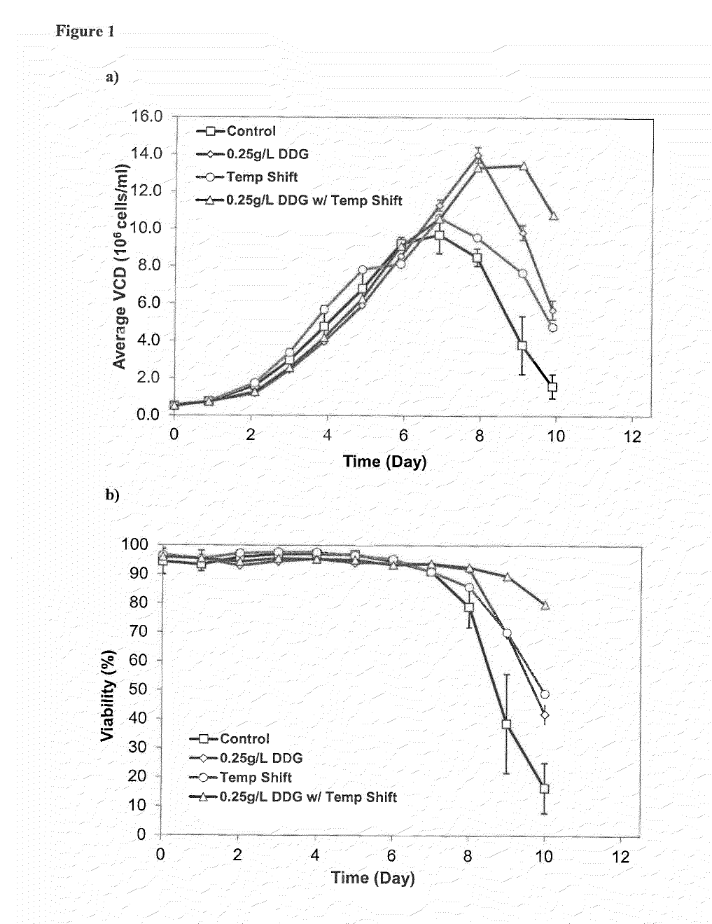

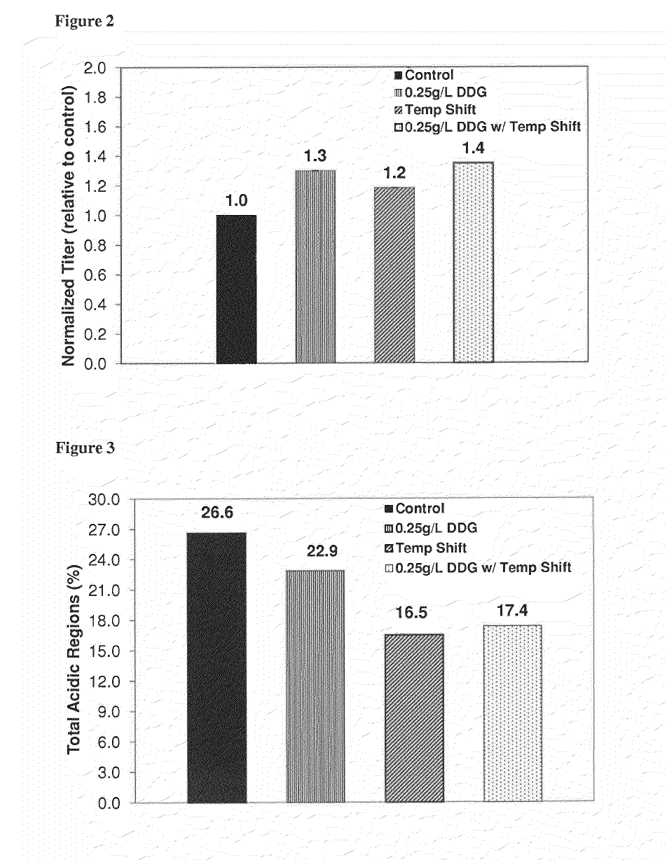

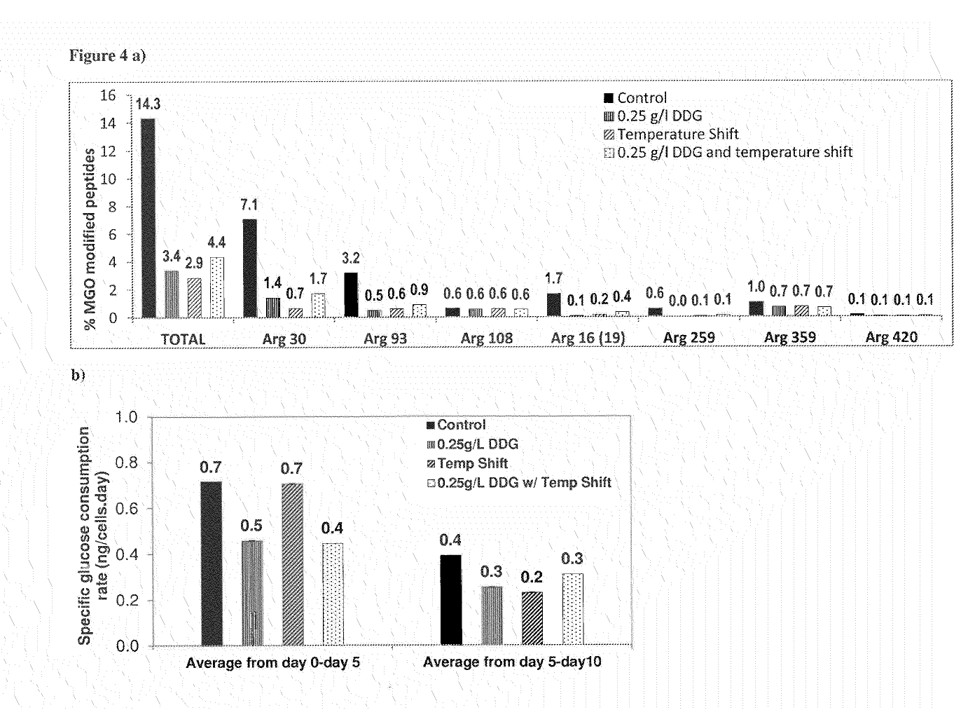

The cells were grown to maximum viable cell densities (VCD) in the range of 10-14×10⁶ cells/ml for the different conditions tested. A slight increase in the cell growth and viability profile was observed in cultures supplemented with 2DDG. Similarly, the cultures that underwent a temperature shift also had a slightly improved viability profile in comparison to that Observed for the control (FIGS. 1(a) and 1 (b)). On Day 10 of culture, the bioreactor cultures for each of the conditions were harvested. The harvest viabilities for all the test conditions were higher than the viability of the control. Samples were subsequently analyzed using Poros A for titer and WCX-10 post protein A purification. Based on the WCX-10 chromatograms, the percentages of total peak(s) area corresponding to the acidic species were quantified. The harvest titers were higher for all the test conditions (20-40% higher than the control titer, FIG. 2). The percentage of acidic species in the control sample (without 2DDG supplementation or temperature shift) was 26.6%. A decrease in acidic species corresponding to 2DDG supplementation to 22.9% was observed. With temperature shift, a substantial reduction in acidic species was observed: 16.5% (without 2DDG supplementation) and 17.4% (with 2DDG supplementation) (FIG. 3). In addition, Lys-C peptide mapping was performed with the harvest protein A purified samples to estimate the extent of MGO modification. The evaluation is represented as relative percentage of MGO modification in peptides at specific arginine residues that can be modified by MGO for adalimumab. The sum total of the percentage modification for each site is represented as the total MGO modification (FIG. 4a). Similar to the acidic species trend, a significant reduction in % MGO modification is observed from about 14.3% in the control to between 2.9-4.4% in three test conditions. This shift corresponds to a significant reduction in MGO modifications at specific arginine residues such as Arg 30 and Arg 93 that have been identified as sites of more prevalent MGO modification.

To assess if this reduction in MGO modification in the test conditions also corresponded to lower glucose consumption, the specific glucose consumption rate was calculated across the two phases of culture: average over day 0-day 5 and average over day 5-10 (period post temperature shift) (FIG. 4b). The specific glucose consumption rate in the conditions with 2DDG supplementation was lower in both phases. In the case of the temperature shift sample, the specific glucose consumption rate was comparable to that of the control for the period before the temperature shift, as expected. In the period post the temperature shift, up to 50% reduction in specific glucose consumption rate relative to that in the control was observed. Thus, a clear correlation has been observed between reduced glucose consumption and the extent of MGO modification of the peptides.

### Example 3

### Effect of Combination of 2DDG, Temperature Shift, Arginine Supplementation, and Lower pH in 3 L Bioreactors with Cell Line

PCT patent application (WO 2013158275A1) reported the use of arginine supplementation and lower pH as approaches to reduce acidic species and MGO modification. WO 2013158275A1 is hereby incorporated by reference into this disclosure. In this study, the combination of 2DDG and temperature shift, evaluated in Example 2, was evaluated with and without arginine supplementation or lower pH. The objective was to evaluate the additive or synergistic effect of these variables on MGO protein modification as well as acidic species of the protein. The experimental design is shown below in Table 1.

TABLE 1
Experimental design for the DOE experiment

| | | | 2DDG | Arginine |
|---|---|---|---|---|
| | | | Concentration | Concentration |
| Number | Final pH | Final Temp | (g/I) | (g/l) |
| 1 | 6.8 | 33 | 0.25 | 0 |
| 2 | 6.8 | 33 | 0 | 4 |
| 3 | 6.8 | 35 | 0 | 0 |
| 4 | 6.8 | 33 | 0.25 | 4 |
| 5 | 7.1 | 33 | 0.25 | 4 |
| 6 | 7.1 | 33 | 0.25 | 0 |
| 7 | 6.8 | 33 | 0 | 4 |
| 8 | 7.1 | 35 | 0 | 4 |
| 9 | 7.1 | 33 | 0 | 0 |
| 10 (control) | 7.1 | 35 | 0 | 0 |
| 11 | 7.1 | 35 | 0.25 | 0 |
| 12 | 6.8 | 35 | 0.25 | 0 |

In this experiment, the cultures were performed in 3 L bioreactors in batch format (DO—30%, 200 rpm) with only glucose feed as described in the materials and methods. The control condition was run at a constant temperature of 35° C., constant pH of 7.1 without any supplementation of 2DDG or arginine (condition #10). Temperature shift was implemented in some tests from 35° C. to 33° C. on day 5 of culture (conditions represented by a final temperature of 33° C. in Table 1). pH ramp was implemented in some conditions from pH 7.1 to pH 6.8 in 3 days of culture post inoculation (conditions represented by a final pH of 6.8 in Table 1).

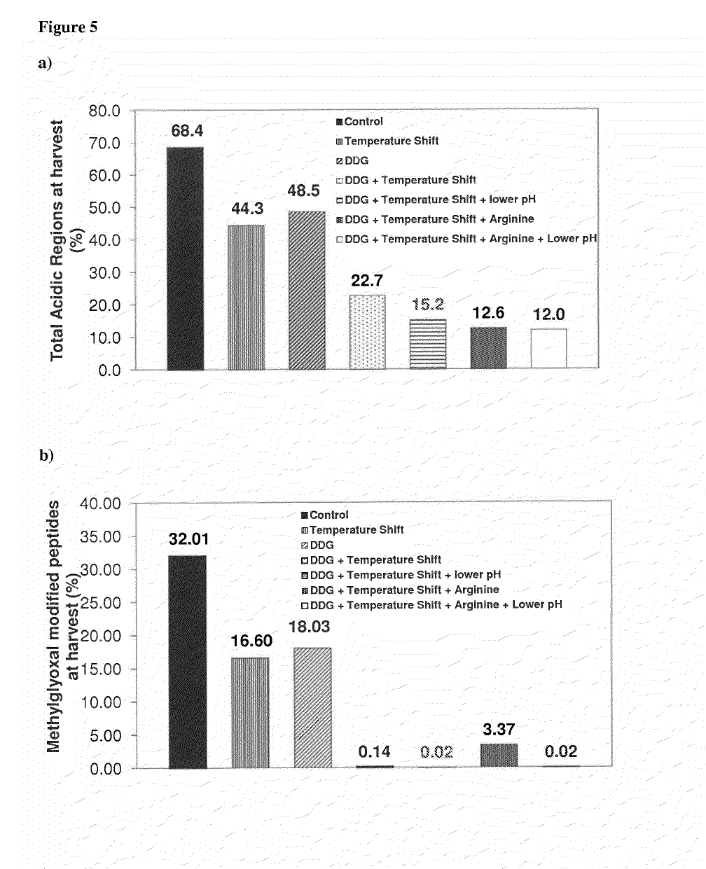

The bioreactor cultures for the different conditions were run for 11 days with daily cell culture supernatant samples (post centrifugation) were collected on days 7 and 8 for extracellular MGO quantification as described in the materials and methods section. On day 11, the cultures from all conditions were harvested and subsequently processed through protein A purification. Protein A eluates were then analyzed using WCX-10 analysis for acidic species quantification (FIG. 5a) and Lys-C peptide mapping for quantification of MGO protein modification (FIG. 5b). The percentage of acidic species in the control sample (condition #10) was as high as 68.4%. A decrease in acidic species corresponding to temperature shift to 44.3% and corresponding to 2DDG supplementation to 48.5% was observed. With the combination of 2DDG supplementation and temperature shift, the percentage of acidic species was significantly reduced to 22.7%. This result supports the observation in Example 2.

Further, in addition to 2DDG supplementation and temperature shift, reducing the final bioreactor culture pH and the additional supplementation of arginine further reduced the percentage of acidic species obtained. The percentage of acidic species were: 15.2% for the combination of 2DDG, temperature shift and lower pH, 12.6% for the combination of 2DDG, temperature shift and arginine supplementation and 12.0% for the combination of 2DDG, temperature shift, lower pH and arginine supplementation, A similar result was observed for the MGO modification that is reduced from 32.01% (control) to 16.60% (temperature shift) and 18.03% (2DDG supplementation). A substantial reduction in MGO modification was observed with the conditions where with 2DDG and temperature shift were combined with arginine supplementation and/or lower pH. With the condition where 2DDG supplementation, temperature shift, lower pH and arginine supplementation were all combined, the MGO protein modification was surprisingly reduced to insignificant amounts of 0.02%. Thus, the use of these approaches in suitable combinations offers significant benefit to maximize reduction of MGO modification of proteins, such as adalimumab.

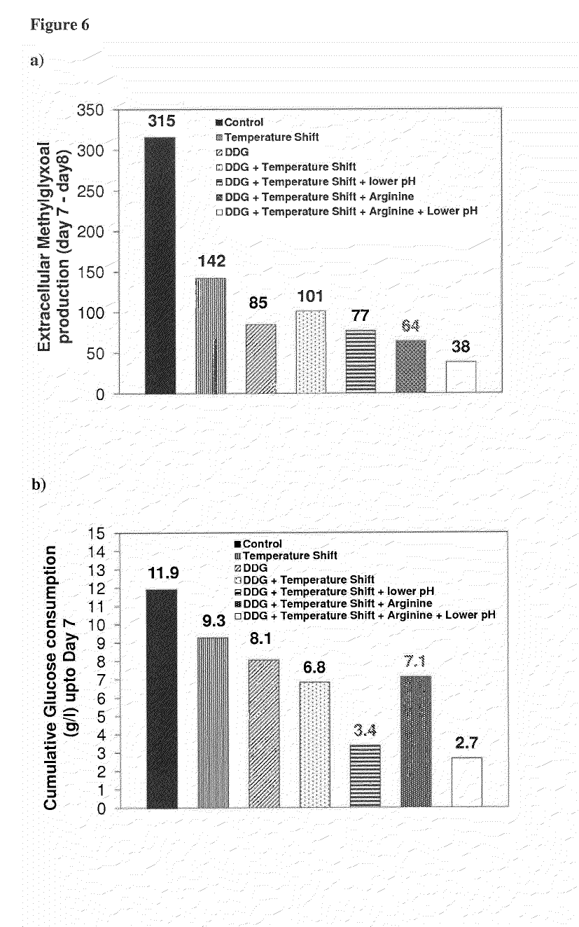

To evaluate how the results observed in this study with respect to product quality correlated to synthesis of MGO byproduct by the cells, measurements were made on the extracellular MGO concentration over the time-course of the culture. The measurements reported here are for days 7-8 when the cell viabilities in all cultures were still significantly high (>75%). This was done to minimize the influence of low viability culture environment on the analysis i.e. the potential scavenging influence of intracellular proteins on MGO. The increase in MGO levels from day 7 to day 8 for the different experimental conditions is reported in FIG. 6a). The rate of MGO production in the control was 315 μM/day and in the condition with the combination of all four parameters (condition #4 in table 1) was 38 μM/day. This correlates well with the estimates of MGO protein modification reported earlier. Thus, the measurement of extracellular MGO early in the culture provides an early indication on the extent of MGO modification of protein. This measurement can also be used as a method to control the MGO modification by altering the culture depending on the levels of MGO measured. For example, control of glucose consumption can be employed as a control strategy to minimize the levels of extracellular MGO/MGO modification.

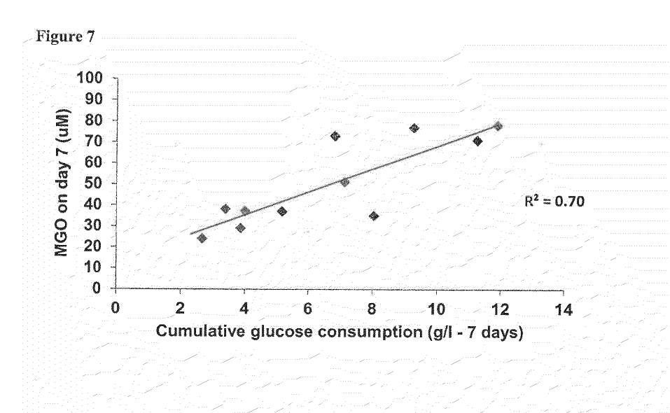

To further assess the applicability of this strategy, the cumulative glucose consumption levels were also evaluated for these conditions in this study (FIG. 6b). This was calculated by using daily glucose concentration measurements via the offline analyzer and by accounting for glucose feeds when added. The cumulative glucose consumption over the first 7 days of culture was 11.9 g/l for the control and 2.7 g/l for condition #4 (combination of all four parameters). This corresponded well to the MGO extracellular production rate from day 7 to day 8 and also to the MGO modification of protein observed for these conditions. Clearly, a significant reduction in glucose consumption was observed both with 2DDG supplementation and temperature shift in this study, as also illustrated in Example 2. In addition, bioreactor culture at lower pH further resulted in reduced glucose consumption. However, no additional reduction in glucose consumption was observed with arginine supplementation (FIG. 6b). A good correlation between the glucose consumption and MGO production on day 7 was observed for the conditions evaluated in the DOE study with an overall R² of 0.70 (FIG. 7). The correlation was observed independent of the strategies used within a specific condition, which indicates the potential breadth of its applicability, i.e., other methods to reduce glucose consumption may also be used for control of MGO.

### Example 4

### Effect of Reduced Glucose Levels and 2DDG Supplementation in 3 L Bioreactors with Cell Line 2

The objective of this experiment was to further evaluate the control of MGO synthesis/MGO protein modification through reduced glucose consumption in a different cell line, Cell line 2. There were two parts to this experiment. In one part, different levels of 2DDG were supplemented (0.05 g/l, 0.15 g/l) similar to the experimental design in Example 2. Prior to this experiment, a study was performed in shake flask culture to evaluate the dose effect of 2DDG on this cell line (data not shown). In the other part, different glucose profiles were maintained in the culture through the feedback control of glucose feed amounts. In this experiment, the cultures were performed in 3 L bioreactors in batch format (pH 7.1, Temperature—36° C., DO—30%, 200 rpm) with only glucose feed as described in the materials and methods (amounts adjusted to target specific glucose concentration levels in the reactor). No temperature shift, pH ramp or arginine supplementation was utilized in any condition in this study. As described in Example 3, extracellular MGO quantification was performed for day 7, 8 and 9 for the samples below and percentage of acidic species were quantified using WCX-10 analysis post protein A of cell culture harvests.

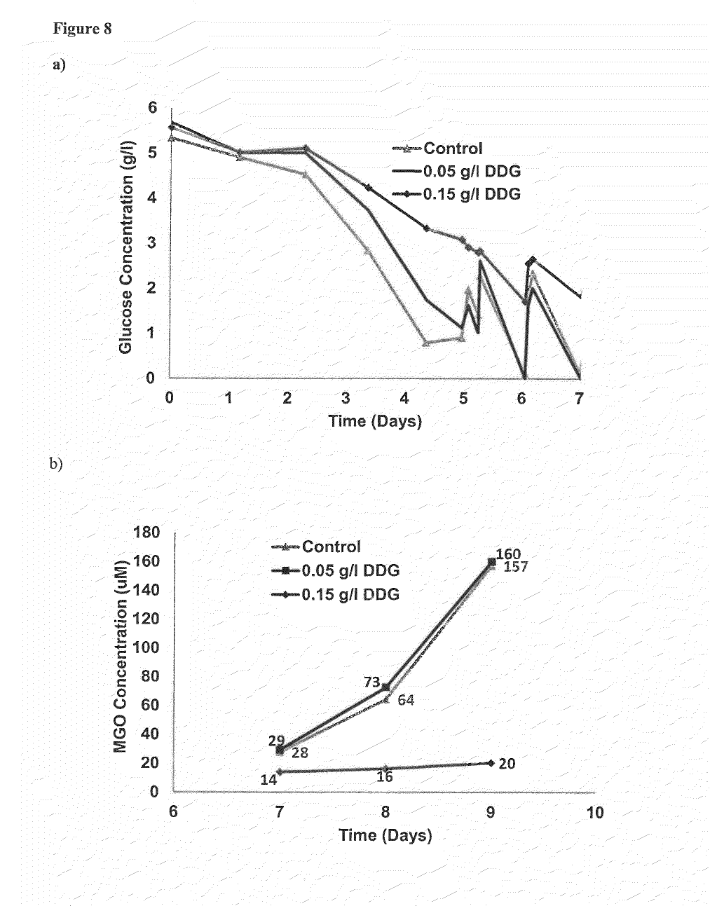

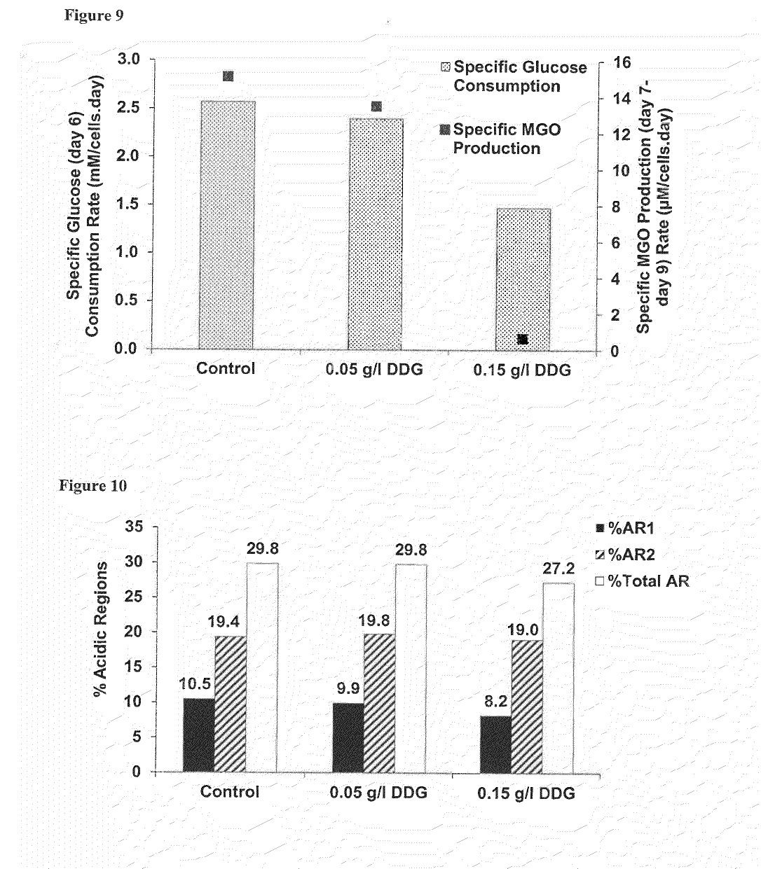

In the first part of the experiment, the comparison of performance was between a control (no 2DDG supplementation) and conditions with different concentrations of 2DDG supplemented (FIGS. 8, 9, and 10). The glucose concentration profile for these three conditions is presented in FIG. 8a. With 2DDG supplementation at 0.15 g/l, a reduction in glucose consumption was observed. The glucose consumption rate, indicated by the slope of the glucose concentration profile, was not significantly lower for the condition with 0.05 g/l 2DDG supplementation as compared to that for the control. Consistent with the glucose consumption results, a substantial reduction in MGO concentration profile was observed relative to that for the control (FIG. 8b). The MGO concentration levels in the control condition were: 28 μM (day 7), 64 μM (day 8), and 157 μM (day 9). The MGO concentration levels in the 0.15 g/l 2DDG condition were reduced to 14 μM (day 7), 16 μM (day 8), and 20 μM. No significant difference between MGO concentration profiles of the control and the 0.05 g/l 2DDG condition was observed. This result was not completely unexpected given the similarity in the glucose consumption rates between these two conditions. A clear correlation was observed between the specific glucose consumption rate (calculated by the ratio of glucose consumption to the viable cell density) on day 6 and specific MGO production rate (calculated as the ratio of extracellular MGO production to the viable cell density) from days 7 to day 9 (FIG. 9). A reduction in percentage of acidic species of the protein was also observed from 29.8% (control) to 27.2% (0.15 g/l 2DDG condition). This reduction correlated mainly with a reduction in % AR1 fraction of the acidic species, a fraction where the majority of MGO modification variants are expected to elute at in a WCX-10 chromatogram.

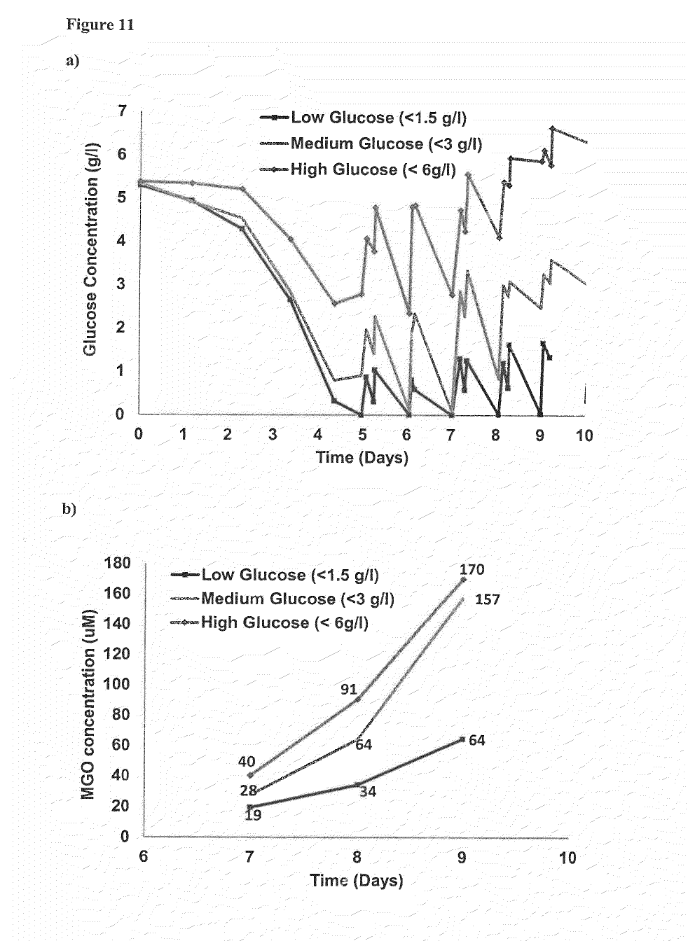

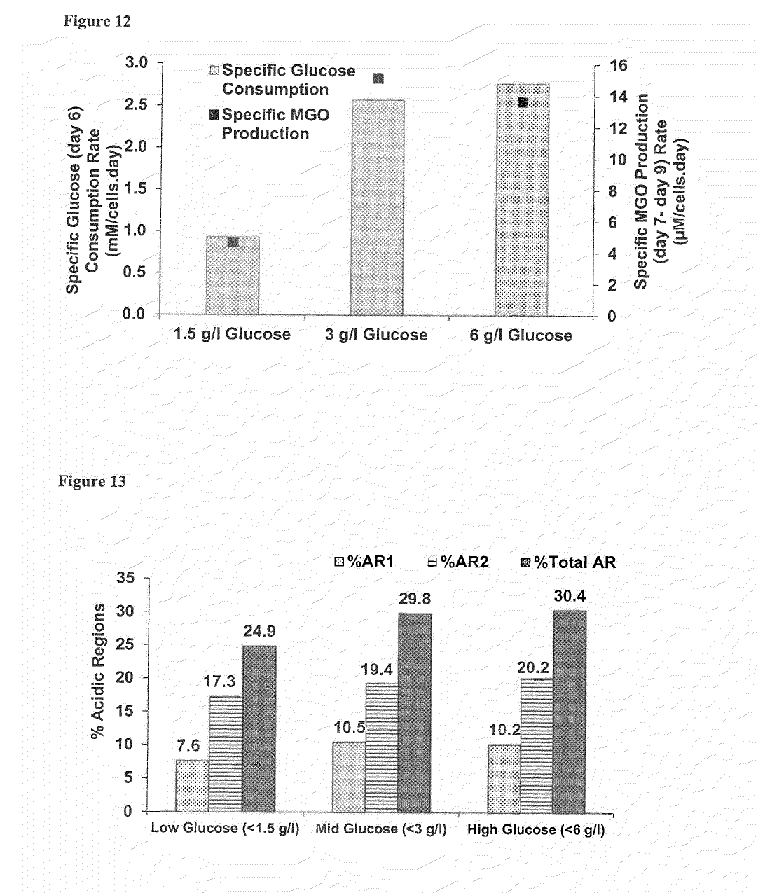

In the second part of the experiment, a similar such comparison of performance was between conditions where glucose feeds were added to target a concentration of 1.5 g/l, 3 g/l and 6 g/l in the cell culture, i.e., to maintain different glucose levels in the bioreactor (FIGS. 11, 12, and 13). The glucose concentration profile for these three conditions is presented in FIG. 11a. Clearly, a different glucose concentration profile was maintained between the different conditions. Consistent with the glucose concentration profiles, a reduction in MGO concentration profile was observed relative to the lower glucose concentration profiles was observed (FIG. 8b). Especially, in the condition with low glucose (˜1.5 g/l), a substantial reduction in the MGO concentration profile was observed. The MGO concentration levels in the high glucose condition (˜6 g/l) were: 40 μM (day 7), 91 μM (day 8), and 170 μM (day 9). The MGO concentration levels in the low glucose condition (˜1.5 g/l) were reduced to 19 μM (day 7), 34 μM (day 8), and 64 μM. In addition, a clear correlation was observed between the specific glucose consumption rate (calculated by the ratio of glucose consumption to the viable cell density) on day 6 and specific MGO production rate (calculated as the ratio of extracellular MGO production to the viable cell density) from days 7 to day 9 (FIG. 12). A reduction in percentage of acidic species of the protein was also observed from 30.4% (high glucose) to 24.9% (low glucose). This reduction also correlated mainly with a reduction in % AR1 fraction of the acidic species, a fraction where majority of MGO modification variants are expected to elute at in a WCX-10 chromatogram.

Thus, this experiment further confirms the reduction of MGO synthesis in cell culture and MGO modification of protein through control of glucose consumption. As illustrated in this example, this strategy works for a second cell line as well.

### Example 5

### Effect of Supplementation of Amino Acid Serine, Vitamin B6 and a Cocktail of Vitamins (Vitamin B2, Vitamin B12, Nicotinamide, Folate, and Choline) in Shake Flask Experiment with Cell Line 2

The objective of this experiment was to test the control of MGO synthesis/MGO protein modification through an altered favorable metabolic state with higher glutathione and cysteine and lower or reduced homocysteine. The supplements serine, vitamin B6 and the cocktail of the listed vitamins were supplemented to potentially unlock the bottleneck at the homocysteine step of the transulfuration pathway. As a consequence of these supplements, it was hypothesized that homocysteine conversion to cysteine would be enabled, thereby providing for more cysteine to convert to glutathione. As mentioned in the introduction section, MGO elimination through the glyoxalase pathway is closely related to the availability of glutathione. Therefore, with the addition of these supplements, a reduction in MGO metabolite as well as protein modification was expected. In this experiment, the cultures were performed in shake flasks in batch format (Temperature—36° C., CO₂—5%) with only glucose feed supplemented from time to time. The experimental design is listed below in Table 2.

TABLE 2
Experimental design (all concentrations represent supplemented amounts in g/l)

| Conditions | Serine | Vitamin B6* | Folate | Choline | Vitamin B12 | Vitamin B2 | Nicotinamide |
|---|---|---|---|---|---|---|---|
| Control | 0 | 0 | 0 | 0 | 0 | 0 | 0 |
| Test 1 | 2 | 0 | 0 | 0 | 0 | 0 | 0 |
| Test 2 | 2 | 0.021 | 0 | 0 | 0 | 0 | 0 |
| Test 3 | 2 | 0 | 0.044 | 0.105 | 0.019 | 0.003 | 0.122 |
| Test 4 | 2 | 0.021 | 0.044 | 0.105 | 0.019 | 0.003 | 0.122 |

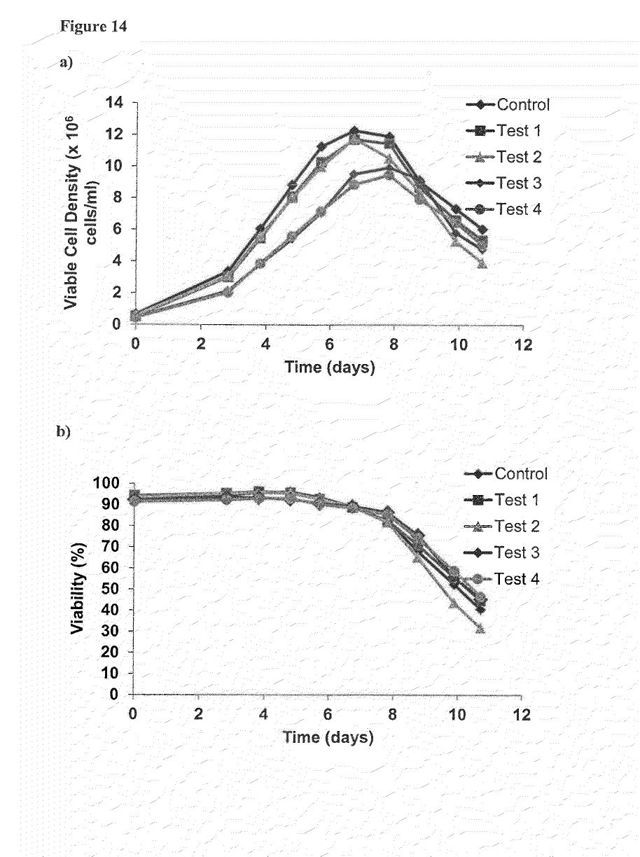

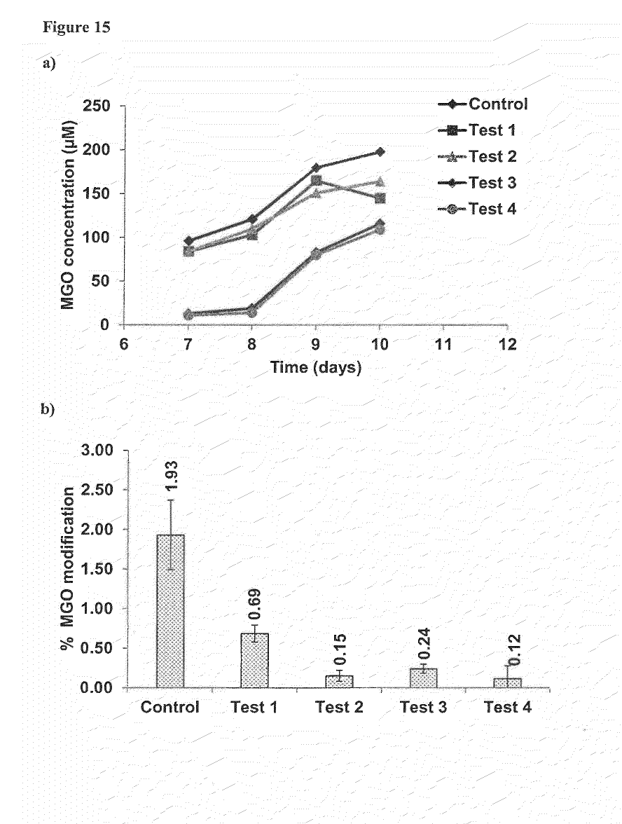

The cells were grown to maximum viable cell densities (VCD) in the range of 10-12×10⁶ cells/ml for the different conditions tested. A slight slower cell growth was observed in Test 3 and Test 4 conditions, relative to the control (FIG. 14a). The viability profiles were comparable between the conditions (FIG. 14b). Cell culture supernatants were collected on days 7, 8, 9, and 10 for extracellular free MGO measurements (FIG. 15a). A significant reduction in the MGO profile was observed for all the test conditions relative to the control. This reduction was most significant for Test 3 and Test 4. On day 11 of culture, the shake flask cultures for each of the conditions were harvested. Samples were then subsequently analyzed using Lys-C peptide mapping for quantification of MGO modification post protein A purification. The evaluation is represented as relative percentage of MGO modification in peptides at specific arginine residues that can be modified by MGO for adalimumab. The sum total of the percentage modification for each site is represented as the total MGO modification (FIG. 15b). The MGO modification in the control was 1.93%. In the condition with serine supplementation alone (Test 1), MGO modification was reduced to 0.68%. In the conditions where the vitamins were supplemented in combination with serine (Tests 2, 3, and 4), the MGO modification was reduced to 0.15% (Test 2), 0.24% (Test 3), and 0.12% (Test 4), respectively.

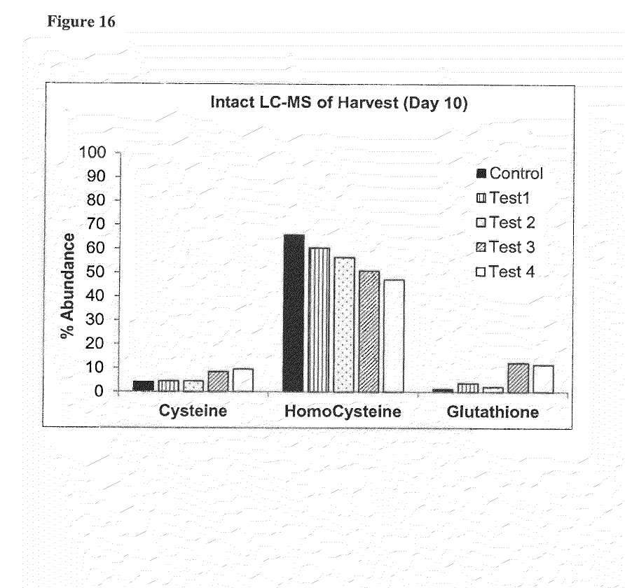

To evaluate the correlation of this reduction in MGO in the test conditions with differences in levels of homocysteine, cysteine and glutathione, the levels of these metabolites were estimated using the LC/MS analysis of cell free supernatant as described in the materials and methods section of Example 1 (FIG. 16). Relative to the control, the homocysteine levels were relatively reduced in all the test conditions. Slightly higher cysteine and glutathione levels were also observed, particularly in the Test 3 and Test 4 conditions, relative to the levels observed in the control. Thus, these results support the use of an altered favorable metabolic state with higher glutathione and cysteine and lower or reduced homocysteine to control MGO synthesis/MGO protein modification.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of this disclosure and the claims.

### REFERENCES

The contents of all cited references (including literature references, patents, patent applications, and websites) that may be cited throughout this application or listed below are hereby expressly incorporated by reference in their entirety for any purpose into the present disclosure. The disclosure may employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the references cited in the instant disclosure.
1. Chaplen, Cytotechnology 26: 173-183, 1998.
2. Kingkeohoi et al., Cytotechnology 48: 1-13, 2005.
3. Dhar et al., J Chromatography 877:1093-1100, 2009.
4. Chaplen, Anal Biochemistry 238:171-178, 1996.
5. Chumsae, C. et al., Anal. Chem. 85:11401-11409, 2013.
6. Chaplen et al., Cytotechnology 22: 33-42, 1996.
7. Paoli et al., Biotechnology and Bioengineering 107: 182-189, 2010.
8. Chaplen et al., PNAS 95: 5533-5538, 1998.
9. Bento et al., PLoS One 5(9) e13007, 2010.

What is claimed is:

1. A method for reducing methylglyoxal (MGO) modification in adalimumab produced by cell culture in chemically defined media, comprising reducing the rate of glucose consumption during cell culture.

2. The method of claim 1, wherein said reduction in the rate of glucose consumption comprises supplementing the cell culture media with a glycolysis inhibitor.

3. The method of claim 1, wherein MGO levels in said cell culture are determined using reverse phase HPLC.

4. The method of claim 2, wherein said glycolysis inhibitor comprises 2-Deoxy-D-Glucose (2DDG).

5. The method of claim 4, wherein said 2DDG is at a concentration of from 0.05 g/l to 0.5 g/l.

6. The method of claim 4, wherein said 2DDG is at a concentration of from 0.15 g/l to 0.25 g/l.

7. The method of claim 1, wherein said reduction in the rate of glucose consumption comprises controlling the concentration of glucose during cell culture.

8. The method of claim 7, wherein said concentration of glucose ranges from 0 to about 2 g/l.

9. The method of claim 2, further comprising reducing the temperature of the cell culture to about 33° C. to about 35° C.

10. The method of claim 9, wherein said reduction in temperature occurs after day 3 of the cell culture.

11. The method of claim 9, wherein said reduction in temperature occurs on day 5 of the cell culture.

12. A method for reducing methylglyoxal (MGO) modification in adalimumab produced by cell culture in chemically defined media, comprising supplementing the cell culture with the amino acid serine.

13. The method of claim 12, wherein the concentration of serine in said cell culture is in the range of 0.1-2 g/l.

14. A method for reducing methylglyoxal (MGO) modification in adalimumab produced by cell culture in chemically defined media, comprising supplementing the cell culture with at least one vitamin selected from the group consisting of vitamin B6, vitamin B2, vitamin B12, Nicotinamide, folate, and choline, and combinations thereof.

15. The method of claim 14, further comprising supplementing the cell culture with the amino acid serine.

16. The method of claim 15, wherein the concentration of serine in said cell culture is in the range of 0.1-2 g/l.

17. A method for reducing methylglyoxal (MGO) modification in adalimumab produced by cell culture in chemically defined media, comprising supplementing the cell culture media with a glycolysis inhibitor and the amino acid arginine, and lowering the pH of said cell culture to a value between 6.0 and 6.9.

18. The method of claim 17, wherein said glycolysis inhibitor is 2-Deoxy-D-Glucose (2DDG).

19. The method of claim 18, wherein said 2DDG is at a concentration of from 0.05 g/l to 0.5 g/l.

20. The method of claim 18, wherein said 2DDG is at a concentration of from 0.15 g/l to 0.25 g/l.

21. The method of claim 17, further comprising reducing the temperature of the cell culture to about 33° C. to about 35° C.

22. The method of claim 21, wherein said reduction in temperature occurs after day 3 of the cell culture.

23. The method of claim 21, wherein said reduction in temperature occurs on day 5 of the cell culture.
